(19) United States
(12) Patent Application Publication
(10) Pub. No.: US 2018/0284116 A1
(43) Pub. Date: Oct. 4, 2018

(54) ANTIGEN POLYPEPTIDE FOR DETECTING PLASMA IMMUNE MARKER-VEGFR1 AUTOANTIBODY AND APPLICATION THEREOF

(71) Applicant: Qingdao Hailanshen Biotechnology Co., Ltd., Qingdao (CN)

(72) Inventors: Jun Wei, Shenzhen (CN); Weili Wang, Shenzhen (CN)

(73) Assignee: Qingdao Hailanshen Biotechnology Co., Ltd., Qingdao (CN)

(21) Appl. No.: 15/524,432

(22) PCT Filed: Aug. 7, 2015
(86) PCT No.: PCT/CN2015/086348

§ 371 Date: May 4, 2017

(30) Foreign Application Priority Data
Nov. 4, 2014 (CN) 201410615968.5

(51) Int. Cl. G01N 33/564

(52) U.S. Cl. CPC G01N 33/564

(57) ABSTRACT

The present application provides a VEGFR1 antigen polypeptide containing three polypeptides, namely H-DEGVYHCKATNQKGSVESSAYLTVQGTSDKSNLE-OH, H-DLKLSCTVNKFLYRDVTWILLRTVNNRTMHYSI-OH and H-ESGLSDVSRP SFCHSSCGHVSEG KRRFTYDHAEL-OH, and an application of the antigen polypeptide to detection of a serum immunity marker, namely a VEGFR1 autoantibody. The present invention also provides a method for detecting the serum immunity marker-VEGFR1 autoantibody by using the VEGFR1 antigen polypeptide described in the present application and a test kit containing the VEGFR1 antigen polypeptide.

### CROSS-REFERENCE TO RELATED APPLICATIONS

This application is a National Stage of International Application No. PCT/CN2015/086348, filed on Aug. 7, 2015, which claims priority to Chinese Patent Application No. 201410615968.5, filed on Nov. 4, 2014, both of which are hereby incorporated by reference in their entireties.

### SEQUENCE LISTING

The instant application contains Sequence Listing which has been submitted in ASCII format via EFS-Web and is hereby incorporated by reference in its entirety. Said ASCII copy, created on May 2, 2017, is named 04305571.txt and is 1.38 KB in size.

### TECHNICAL FIELD

The present invention belongs to the technical field of immunology, and relates to a VEGFR1 antigen polypeptide. Using the antigen polypeptide can prepare a specific reagent for detecting VEGFR1 antibody level in human plasma.

### BACKGROUND ART

The recent studies have shown that discovering and determining tumor-associated antibody in blood of healthy people has the value of instructing targeted treatment. For example, Trastuzumab and Bevacizumab which have been broadly used in the clinic take epidermal growth factor receptor-2 (HER2) and vascular endothelial growth factor A (VEGF-A) as targets, which become an importance means of the clinical treatment for advanced stage tumor. However, since the side effects are too great, these two monoclonal antibodies can only be used as third line treatment medicine. Thus, developing a new means used for preventing tumor metastasis and recurrence after local treatment while having low side effects is a present problem urgently needed to solve. But in the field of immune treatment, the lead technical barrier for solving this problem is to find an innovative practical solution for target technology, then a brand new tumor immune treatment solution can be further developed.

VEGFR1 is also referred to as vascular endothelial growth factor receptor-1, and has been considered as one of specific proteins expressed by cancer cells. A large number of studies have found that many human solid tumors can express VEGFR1 abundantly, such as liver cancer, lung cancer, kidney cancer, intestinal cancer, breast cancer and so on. Because VEGFR1 antigen epitope polypeptide is mainly located on the surface of tumor cell and can be used as target spot for corresponding antibody killing tumor, the medical field universally acknowledges that the clinical application of corresponding antibody of VEGFR1 is very likely to become a brand new anticancer treatment means which is achieved fastest and has low side effects. Currently, in the field of biotechnology, the only way is to carry out the detection and screening of corresponding antibody of VEGFR1 using recombinant protein as antigen, which will go through vector construction, transfection, expression, screening, purifying and other tedious processes. However, since the space structure of protein is complicated and linear antigen epitope is hardly to be exposed, specificity of the antibody detected by using recombinant protein is poor, and a considerable proportion of the antibody cannot be combined with antigen target spot on the surface of tumor cell membrane, as a result, only substandard qualitative detection means can be carried out, and the practical means for tumor prevention and treatment cannot be developed accordingly. Meanwhile, high sensitivity of ELISA method has an extreme high requirement for stability of protein purification technology, therefore, the recombinant protein meeting the requirement of detection is costly. There is as yet no effective means for detection and screening of the corresponding antibody of VEGFR1 at home and abroad.

The linear VEGFR1 antigen epitope polypeptide specifically designed by the present invention can effectively solve the above-mentioned technical problems, prepare detection reagent with extremely high specificity, detect the index value of concentration of the immune marker—VEGFR1 antibody in the plasma of healthy blood donors according to set process specification, and obtain package solutions for the detection, identification, qualitative and quantitative applications of VEGFR1 antibody with high accuracy, simple operation and moderate cost. Thus, the technology of the present invention can lay a realistic basis for developing a brand new tumor immune treatment solution with low side effects using natural VEGFR1 antibodies in healthy human body.

### SUMMARY OF THE INVENTION

The object of the present invention is to provide a linear antigen polypeptide for detecting VEGFR1 autoantibody, and thus to prepare a specific reagent for detecting VEGFR1 autoantibody, and design a corresponding preferably implemented operation process scheme. The present invention totally designs three VEGFR1 linear antigen polypeptides, whose amino acid sequences are shown in Table 1.
Table 1. VEGFR1 antigen polypeptide sequence

### Abbreviations VEGFR1 H-DEGVYHCKATNQKGSVESSAYLTVQGTSDKSNLE-OH

H-DLKLSCTVNKFLYRDVTWILLRTVNNRTMHYSI-OH
H-ESGLSDVSRPSFCHSSCGHVSEGKRRFTYDHAEL-OH

It is well known that, since the combination of antigen with antibody actually only occurs between the antigenic determinant and antibody binding site. More fully complementary space structure and configuration of antigen and antibody lead to more stable binding of the antigen and the antibody, stronger specificity and higher binding efficiency. Since a target antibody and a binding site structure thereof are prerequisites, the binding state and affinity characteristics of the whole protein antigen and the antibody can be determined according to the antigenic determinant.

Accordingly, based on the biological characteristics of VEGFR1 protein, the immunoinformatics forecast and simulation regarding to multiple epitopes, and analysis of various parameters related to antigenicity, the present invention designs the amino acid sequences of the above-mentioned three linear polypeptide antigens which are fully complementary with the target antibody in terms of space structure and configuration.

Numerous studies prove that VEGFR1 is one tumor-associated antigen and can be expressed in different solid tumors. Therefore, VEGFR1 antibody can be considered as a natural anti-tumor antibody and can play the immunological surveillance role in human body and prevent the formation and progression of tumors. The technology of the present invention fills in the blank of quantitative detection of the natural anti-tumor antibody, and provides an important tool to develop new products in a plasma biologicals company and develop new measures against tumors in clinical medicine. For example, the plasma biologicals company can utilize the technology of the present invention to screen plasma to prepare gamma globulin enriched in VEGFR1 antibody for the clinical tumor prevention and treatment. Also, in clinics, patients at early stage of tumor after local treatment (for example, after surgery or radiotherapy) can be directly transfused with VEGFR1 antibody-enriched plasma screened by the technology of the present invention to enhance the immunosurveillance function thereof and prevent the tumor recurrence and metastasis. This is the application value of the technology of the present invention.

As shown in Table 1, the three antigen polypeptides applied to the detection of plasma VEGFR1 autoantibody by ELISA method is implemented in the form of high-purity products with the purity recommended being not less than 95%. The technical effect which can be achieved in the present invention is illustrated by the following technical verification using the sandwich contrast test pattern which is common in the art.

The present application provides a method for detecting plasma immune marker VEGFR1 autoantibody by using the three antigen polypeptides shown in Table 1, comprising the following steps of:

1. before operation, dissolving each of the antigen polypeptides into 5 mg/ml storage solution by using 65%-67% acetic acid, and then isometrically mixing and placing in a refrigerator at −20° C. (error less than 2° C.) for storage;

2. before start-up of the operation, firstly diluting the isometric mixed solution of the three antigen polypeptides shown in Table 1 with a coating buffer to 10-50 mcg/ml, wherein the coating buffer is 0.1 M phosphate buffer solution containing 0.15 M sodium chloride and 10 mM EDTA, and a pH value is 7.2-7.4;

3. coating a maleimide-activated 96-well plate (Thermo Scientific, U.S.A.), incubating at 4° C. overnight, and rinsing with a washing solution for three times, wherein the washing solution is 0.1 M phosphate buffer solution containing 0.15 M sodium chloride and 0.1% TWEEN-20, and a pH value is 7.0-7.4; and

4. performing step-by-step sampling and analysis as follows:

a. setting double holes in to-be-tested plasma sample and setting two negative control holes (NC, a reference being a negative control solution free of plasma VEGFR1 antibody, thereby obtaining index values of the three antigen polypeptides of the present invention shown in Table 1 in the negative environment free of VEGFR1 autoantibody, aiming to prove that the composition of the present invention is unlikely to appear a positive result in the case of having no VEGFR1 autoantibody) and two positive control holes (PC, a reference being a positive control solution containing VEGFR1 antibody standard, thereby obtaining index values of the three antigen polypeptides of the present invention shown in Table 1 at the level of VEGFR1 antibody standard reference content, aiming to prove that the composition of the present invention is likely to appear a positive result in the case of having VEGFR1 autoantibody, and further serving as a baseline for comparison to the index values of the to-be-tested plasma sample);

b. diluting the to-be-tested plasma sample according to 1:200 by using an analysis solution, the analysis solution being the same as the antigen coating solution which is a 0.1 M phosphate buffer solution containing 0.15 M sodium chloride and 10 mM EDTA, and a pH value is 7.0-7.4, adding 100 μl into each hole, incubating at 25° C. for 1-2 h, and then rinsing for three times;

c. after according to the step b, using the analysis solution (which is a 0.1 M phosphate buffer solution containing 0.15 M sodium chloride and 10 mM EDTA, and the pH value is 7.0-7.4), diluting horseradish peroxidase-labeled goat anti-human IgG (for verifying whether a detected substance in plasma is a specific antibody), the antibody having a working range of 1:10000-1:150000, adding 100 μl into each hole, and incubating at 25° C. for 1-2 h;

d. after using the aforementioned washing solution (which is 0.1M phosphate buffer solution containing 0.15M sodium chloride and 0.1% Tween-20, and the pH value of 7.0-7.4) to rinse for three times, adding 100 μl of a mixed solution of 3.3′,5,5′-tetramethylbenzidine (TMB) and peroxidase into each hole, and keeping in dark place for 20-30 min at room temperature; and

e. adding 50 μl of 10% sulfuric acid solution (10% H2SO4) as a stop solution into each hole, then detecting a value of optical density (OD) by using a microplate reader, wherein a detection wavelength is 450 nm and a reference wavelength is 630 nm, completing a detection process within 10 min after the stop solution is added, and accordingly quantitatively analyzing individual plasma VEGFR1 autoantibody level; and

5. performing an analysis on data obtained by the detection for each individual during group random sampling and analysis, and determining the relative level of VEGFR1 autoantibody in the plasma by using a positive sample ratio (PSR), wherein a PSR calculation method is as follows: PSR=[VEGFR1 OD value−NC OD value]/[PC OD value−NC OD value]; and plotting by using a normality quartering method (Q-Q).

Three antigen polypeptides listed in Table 1 can be prepared into a simple and easy-to-use convenient test kit during practical application, which can be vacuum sealed and packaged with nonmetallic materials and can be stored for not less than six months at 4° C. Thus, the present application also provides a test box containing the three antigen polypeptides listed in Table 1. In short, after a maleimide-activated 96-well plate coated with a mixed solution of the three antigen polypeptides is dried in an oven at 45° C., the plate is vacuum sealed and packaged with nonmetallic packaging materials, and each reagent combination serves as a convenience test kit. A suggestion is given that all the three antigen polypeptides are articles not less than 95% in purity.

### BRIEF DESCRIPTION OF DRAWINGS

FIG. 1 is a quartile distribution graph of plasma VEGFR1 antibody concentration.

In the FIGURE, the horizontal ordinate represents the plasma VEGFR1 antibody concentration indicative of PSR value, the longitudinal coordinate represents the quartile distribution range of plasma VEGFR1 antibody concentration, and the PSR value horizontally corresponding to 0 point is a median.

### DETAILED DESCRIPTION OF THE INVENTION

1. Sample collection is as follows: 121 plasma samples of normal adult human are collected, including plasma samples of 65 male cases and 56 female cases. All the blood sample providers are individuals determined by clinical physical examination to be free of having any type of tumors. Before operation, all the plasma samples are stored at −80° C., and verification shows that no plasma sample with repeated freezing and thawing (not more than 3 times) occurs during a storage period not longer than two years.

2. Sample detection is as follows: the plasma samples are thawed at 4° C., and the three antigen polypeptides listed in Table 1 used in the experiment are synthesized by UK SEVERN BIOTECH Co., Ltd., having a purity of 95%, and specific operation steps are as follows:

(1) before operation, dissolving each of antigen polypeptides into 5.7 mg/ml storage solution by using 67% acetic acid, and then isometrically mixing and placing in a refrigerator at −20° C. for storage;

(2) before start-up of the operation, firstly diluting the mixed solution of the three antigen polypeptides with a coating buffer to 28.5 mcg/ml, wherein the coating buffer is 0.1 M phosphate buffer solution containing 0.15 M sodium chloride and 10 mM EDTA, and the measured pH value is 7.2;

(3) coating the maleimide-activated 96-well plate (Thermo Scientific, US), incubating at 4° C. for 16.5 h overnight, and rinsing with a washing solution for three times, wherein the washing solution is 0.1 M phosphate buffer solution containing 0.15 M sodium chloride and 0.1% Tween-20, wherein the measured pH value is 7.3; and

(4) performing step-by-step sampling and analysis as follows:

a. setting double holes in to-be-tested plasma sample and additionally setting two negative control holes (NC, a reference being a human plasma negative control solution free of VEGFR1 antibody and provided by the company Sigma-Aldrich) and two positive control holes (PC, a reference is also a human plasma VEGFR1 antibody standard positive control solution provided by the company Sigma-Aldrich);

b. diluting the plasma according to 1:200 by using an analytic solution, the analytic solution being the same as an antigen coating solution which is 0.1 M phosphate buffer solution containing 0.15M sodium chloride and 10 mM EDTA, wherein the measured pH value is 7.2, adding 100 μl into each hole, and incubating at 25° C. for 1.5 h;

c. after using the aforementioned washing solution (which is 0.1 M phosphate buffer solution containing 0.15 M sodium chloride and 0.1% Tween-20, and the measured pH value is 7.2) to rinse for three times, diluting horseradish peroxidase-labeled goat anti-human IgG (provided by the company Sigma-Aldrich) by using the analytic solution, antibody having an working environment according to 1:285000, adding 100 μl into each hole, and incubating at 25° C. for 1.5 h;

d. after using the washing solution (which is 0.1 M phosphate buffer solution containing 0.15 M sodium chloride and 0.1% Tween-20, wherein the measured pH value is 7.2), rinsing for three times, adding 100 μl of 3,3,5,5-Tetramethylbenzidine (TMB) and a peroxidase mixed solution (provided by Life Technologies company) into each hole, and keeping in dark place for 25 min at room temperature; and

e. adding 50 μl of 10% sulfuric acid solution (10% H2SO4) as stop solution into each hole, then detecting the value of optical density (OD) by using a microplate reader, wherein the detection wavelength is 450 nm and reference wavelength is 630 nm, and completing the detection within 10 minutes after stop solution is added.

3. Result analysis: the comparative analysis of VEGFR1 autoantibody distribution in target individuals is further carried out below in accordance with the experimental results. Upon analysis of the data obtained by the foregoing detection, the level of VEGFR1 autoantibody in plasma is determined by using a positive sample ratio (PSR), and PSR is calculated according to the following formula: PSR=[VEGFR1 OD value−NC OD value]/[PC OD value−NC OD value], and made into the following FIGURE by normal quartering method (Q-Q). In the FIGURE, the horizontal ordinate represents PSR value indicative of plasma VEGFR1 antibody concentration, the longitudinal coordinate represents the quartile distribution range of plasma VEGFR1 antibody concentration, and the PSR value horizontally corresponding to 0 point is the median.

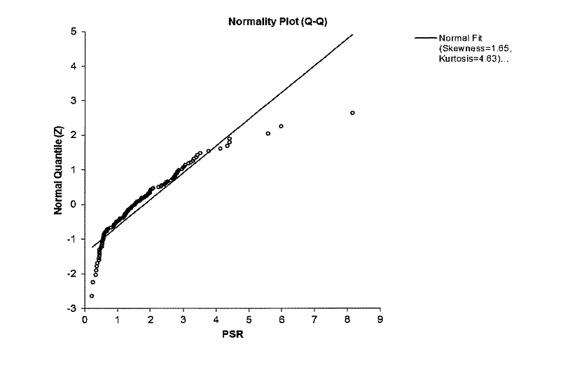

As shown in FIG. 1, the median PSR of plasma VEGFR1 antibody concentration is equal to 1.53, the skewness coefficient S is equal to 1.65, and the kurtosis coefficient K is equal to 4.63, which are statistically highly significant (w=0.89, p<0.0001). In the normal distribution fitting curve, the maximum value of rightward turning point is the threshold value (cut-off=3.8), namely, 8 out of totally 121 healthy adult plasma samples are rich in VEGFR1 autoantibody, and the number of individuals above the threshold value in this embodiment accounts for 6.6%. It can also be seen according to this embodiment that the level of VEGFR1 antibody in randomly selected healthy adult plasma samples is in skew distribution, wherein 8 plasma samples are rich in natural VEGFR1 autoantibody and valuable in clinical application.

In this embodiment, the synchronous negative contrast test proves that no false results exist in the present invention, and in the above FIGURE, the horizontal ordinate represents the calculated values of PSR in individual blood samples, in reference to human plasma VEGFR1 antibody standard as positive control provided by Sigma-Aldrich company, which are marked to generate the above FIGURE. In order to easily understand, in another aspect, the VEGFR1 autoantibody concentration (PSR value) can be distributed in 4 zones, and the proportion of cases falling within each antibody concentration zone in this embodiment is shown in the following table.

Annexed table Distribution of VEGFR1 autoantibody
in 121 healthy adult plasma samples

| Cases | (%) | Antibody Concentration (PSR) |
|---|---|---|
| 34 | (28.1%) | 0.00-0.90 |
| 50 | (41.3%) | 0.91-2.30 |
| 29 | (24.0%) | 2.31-3.79 |
| 8 | (6.6%) | 3.80-8.60 |

In the above specific embodiment, the human plasma VEGFR1 antibody negative control solution and positive standard control solution provided by international authority biochemical reagent company Sigma-Aldrich are used, and the results of the sandwich contrast test prove that: the three linear antigen polypeptides designed in the present invention can specifically bond with VEGFR1 autoantibody in plasma, namely, the amino acid sequences of the three linear polypeptide antigens are effectively complementary with VEGFR1 antibody protein in terms of space structure and configuration, thus quantitatively determining the level of plasma VEGFR1 autoantibody in different individuals. As a result, a practical means for detecting plasma VEGFR1 autoantibody is obtained, and at the same time, the synthesis process for preparation of the three linear polypeptide antigens designed by the present invention is relatively simple and cost-reasonable, which lays a theoretical and practical basis for clinical application and development in tumor treatment with natural VEGFR1 autoantibody-rich plasma.

The foregoing descriptions of specific exemplary embodiments of the present invention have been presented for purposes of illustration and description. They are not intended to be exhaustive or to limit the invention to the precise forms disclosed, and obviously many modifications and variations are possible in light of the above teachings. The exemplary embodiments were chosen and described in order to explain certain principles of the invention and their practical application, to thereby enable others skilled in the art to make and utilize various exemplary embodiments of the present invention, as well as various alternatives and modifications thereof. It is intended that the scope of the invention be defined by the Claims appended hereto and their equivalents.

1. A composition for detecting VEGFR1 autoantibody, comprising the following three antigen polypeptides:
H-DEGVYHCKATNQKGSVESSAYLTVQGTSDKSNLE-OH;
H-DLKLSCTVNKFLYRDVTWILLRTVNNRTMHYSI-OH;
H-ESGLSDVSRPSFCHSSCGHVSEGKRRFTYDHAEL-OH.

2. The composition according to claim 1, wherein the ratio of individual antigen polypeptides in the composition is 1:1:1.

3. A method for detecting VEGFR1 autoantibody in an individual, comprising the following steps: mixing equal amounts of the three antigen polypeptides according to claim 1, then coating a maleimide-activated 96-well plate, incubating at 4° C. overnight, rinsing, and performing step-by-step sampling and analysis.

4. The method according to claim 3, wherein the step-by-step sampling and analysis comprises setting double holes in to-be-tested plasma sample while setting two negative control holes and two positive control holes, diluting plasma with an analysis fluid, diluting horseradish peroxidase-labeled goat anti-human IgG, rinsing, and adding 100 μl of 3,3′,5,5′-tetramethylbenzidine (TMB) and peroxidase mixed solution into each hole, keeping in dark plate for 20-30 min at the room temperature, adding 50 μl of 10% sulfuric acid solution (10% H2SO4) as a stop solution into each hole, and detecting the optical density (OD) value by using a microplate reader, wherein the detected wavelength is 450 nm, and the reference wavelength is 630 nm.

5. (canceled)

6. A test kit set comprising the following three antigen polypeptides:
H-DEGVYHCKATNQKGSVESSAYLTVQGTSDKSNLE-OH;
H-DLKLSCTVNKFLYRDVTWILLRTVNNRTMHYSI-OH;
H-ESGLSDVSRPSFCHSSCGHVSEGKRRFTYDHAEL-OH wherein the polypeptides are independently packaged under vacuum with nonmetallic medical packaging materials.

7. The test kit according to claim 6, wherein the ratio of individual antigen polypeptides in the composition is 1:1:1.


SEQUENCE LISTING (3 sequences)

SEQ ID NO: 1 - protein, 34 aa, ARTIFICIAL SEQUENCE
  SYNTHETIC
DEGVYHCKATNQKGSVESSAYLTVQGTSDKSNLE

SEQ ID NO: 2 - protein, 33 aa, ARTIFICIAL SEQUENCE
  SYNTHETIC
DLKLSCTVNKFLYRDVTWILLRTVNNRTMHYSI

SEQ ID NO: 3 - protein, 34 aa, ARTIFICIAL SEQUENCE
  SYNTHETIC
ESGLSDVSRPSFCHSSCGHVSEGKRRFTYDHAEL